(19) United States
(12) Patent Application Publication
(10) Pub. No.: US 2016/0029907 A1
(43) Pub. Date: Feb. 4, 2016

(54) Radial Check Device

(71) Applicant: PROMEDICA HEALTH SYSTEM, INC., Toledo, OH (US)

(72) Inventor: Periakaruppan Kasi Ramanathan, Ottawa Hills, OH (US)

(73) Assignee: PROMEDICA HEALTH SYSTEM, INC., Toledo, OH (US)

(21) Appl. No.: 14/882,715

(22) Filed: Oct. 14, 2015

Related U.S. Application Data
(63) Continuation-in-part of application No. PCT/US2014/036777, filed on May 5, 2014.
(60) Provisional application No. 61/819,747, filed on May 6, 2013.
(60) Provisional application No. 62/064,503, filed on Oct. 16, 2014.

(51) Int. Cl. A61B 5/026; A61B 5/024; A61B 5/00; A61B 5/1455

(52) U.S. Cl. CPC A61B 5/0261; A61B 5/14552; A61B 5/02427; A61B 5/02438; A61B 5/6826; A61B 5/6824; A61B 5/7445; A61B 5/6838

(57) ABSTRACT

A radial check device and methods are provided for accessing ulnar and/or radial flow and producing documentation of such assessment.

### CROSS-REFERENCE TO RELATED APPLICATIONS

This is a continuation-in-part application of PCT/US 14/36777, filed under the authority of the Patent Cooperation Treaty on May 5, 2014; published; which claims priority to U.S. Provisional Application No. 61/819,747, filed under 35 U.S.C. §111(b) on May 6, 2013. This application also claims priority to U.S. Provisional Application No. 62/064,503, filed under 35 U.S.C. §111(b) on Oct. 16, 2014. The entire disclosures of all the aforementioned applications are incorporated herein by reference for all purposes.

### TECHNICAL FIELD

The invention relates generally to medical devices. More specifically, the invention is a radial check device.

### BACKGROUND OF THE INVENTION

Radial artery access is being increasingly used by physicians for diagnostic and coronary procedures. In preparation for such procedures, the Allen's test (or modified plethysmography) has been used to determine the patency of the radial and ulnar arteries. This is a manual test in which a healthcare professional, usually a nurse, places his or her thumbs over the radial arteries of a patient while the patient is clenching his or her fists. This compresses the radial arteries. The patient is then asked to open his or her hands into a relaxed position. The healthcare professional then observes the color of the palms, which should normally turn pink promptly. An abnormal test occurs when the color of the palm does not return within eight seconds. This procedure is then repeated by occluding the ulnar arteries. In another form of this test, the fingers of the healthcare professional are used to occlude both the radial and ulnar arteries of the patient. Pressure on the ulnar artery is then removed while maintaining pressure on the radial site. The color of the palm is then observed. This procedure is then performed on the other arm.

In those patients that require a second procedure through the same radial site, it is often useful to perform a reverse Allen's test. In this procedure, the healthcare professional releases pressure over the radial artery rather than the ulnar artery. This may detect proximal radial artery disease/occlusion that may be asymptomatic.

Barbeau's test is another preparation procedure. This test includes the steps as follows: placing a pulse oximeter (plethysmography) on an index finger or a thumb to demonstrate a normal waveform/tracing and releasing pressure over the ulnar artery and watching the pulse oximetry tracing. The immediate return of normal waveform suggests a normal (positive) test, which is indicative of good ulnar flow and a lower risk of hand ischemia with radial catheterization. If the waveform does not immediately return, one can wait two minutes to evaluate the waveform. If the waveform returns within two minutes, then one can still consider radial catheterization.

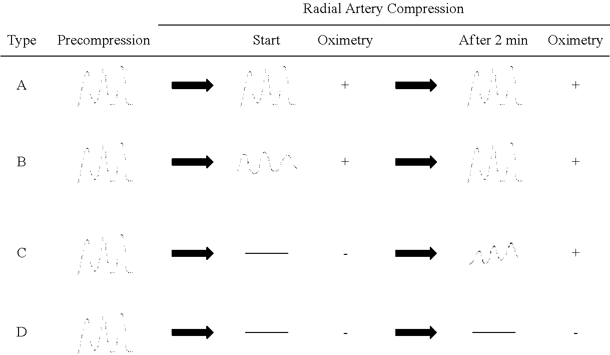

It has been found that these tests are deficient for a variety of reasons. For example, they are done in a subjective fashion in which the healthcare professional uses his or her observation of palm color to determine the patency of the arteries. Further, they require extensive expertise and training on the part of the healthcare professional performing the tests. Finally, they do not provide a permanent record of the tests for future use.

### BRIEF SUMMARY OF THE INVENTION

The invention provides a medical device that overcomes the deficiencies of the current manual tests as described above. In this regard, the radial check device according to the invention establishes a new standard of pre-procedural care for patients undergoing any type of radial artery canalization. Further, the invention provides a relatively simple automated test that generates documentation of ulnar and radial flow.

### BRIEF DESCRIPTION OF THE DRAWINGS

FIG. 1 is a perspective view of an embodiment of the radial check device according to the invention positioned on a patient;

FIG. 2 is a perspective view of an embodiment of the cuff according to the invention;

FIG. 3 is a perspective view of an alternative embodiment of the radial check device according to the invention positioned on a patient;

FIG. 4 is an exploded view of the radial check device shown in FIG. 3;

FIG. 5 is an exploded view of the radial check device shown in FIG. 3;

FIG. 6 is an exploded view of the radial check device shown in FIG. 3;

FIG. 7 is a schematic view of an embodiment of the control system for the radial check device shown in FIG. 3;

FIG. 8A is a schematic view of an embodiment of the control system for the radial check device shown in FIG. 3;

FIG. 8B is a schematic view of an embodiment of the control system for the radial check device shown in FIG. 3;

FIG. 9 is a perspective view of an alternative embodiment of the radial check device according to the invention positioned on a patient;

FIG. 10 is a detailed perspective view of the occlusion cuff and the first and second inflatable portions of the radial check device shown in FIG. 9; and

FIG. 11 is a cross-sectional view taken along line 11-11 of FIG. 10.

### DETAILED DESCRIPTION OF THE INVENTION

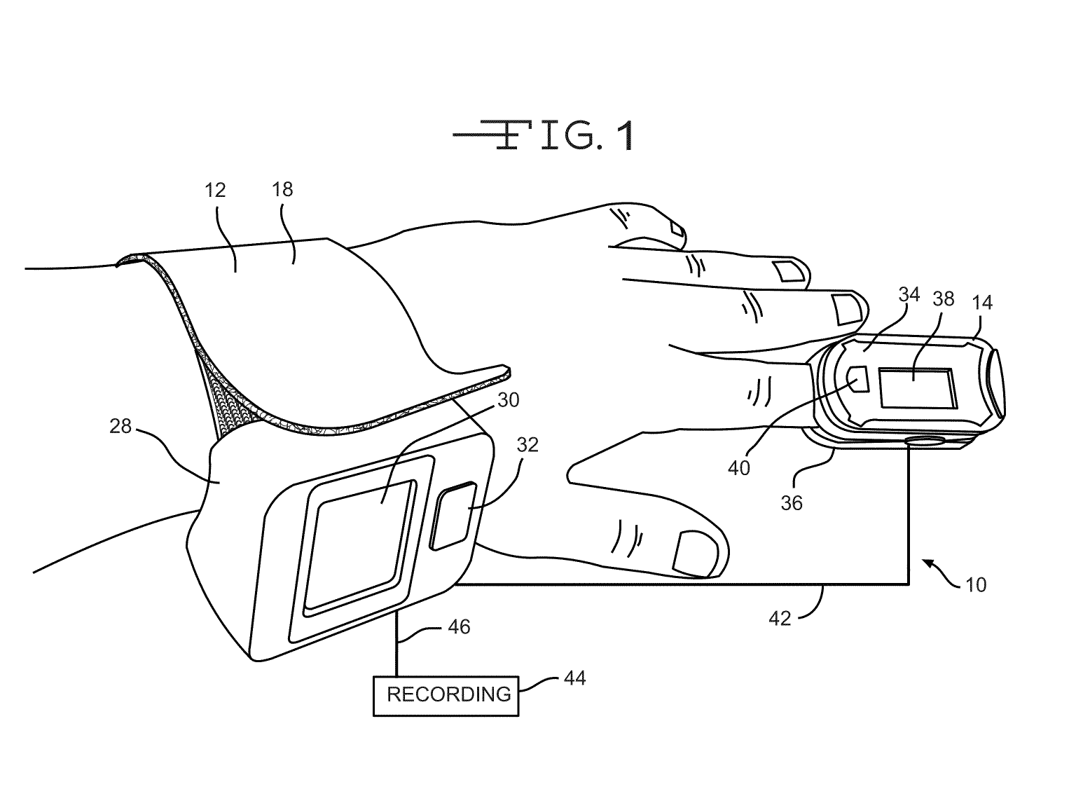

The invention will now be described in detail with reference being made to the drawings. In the drawings, an embodiment of the radial check device according to the invention is indicated generally by the reference number “10.” Referring to FIG. 1, the radial check device 10 has an occlusion cuff 12 configured for positioning on a patient's arm and a pulse oximeter 14 configured for positioning on a patient's finger, such as an index finger, or thumb.

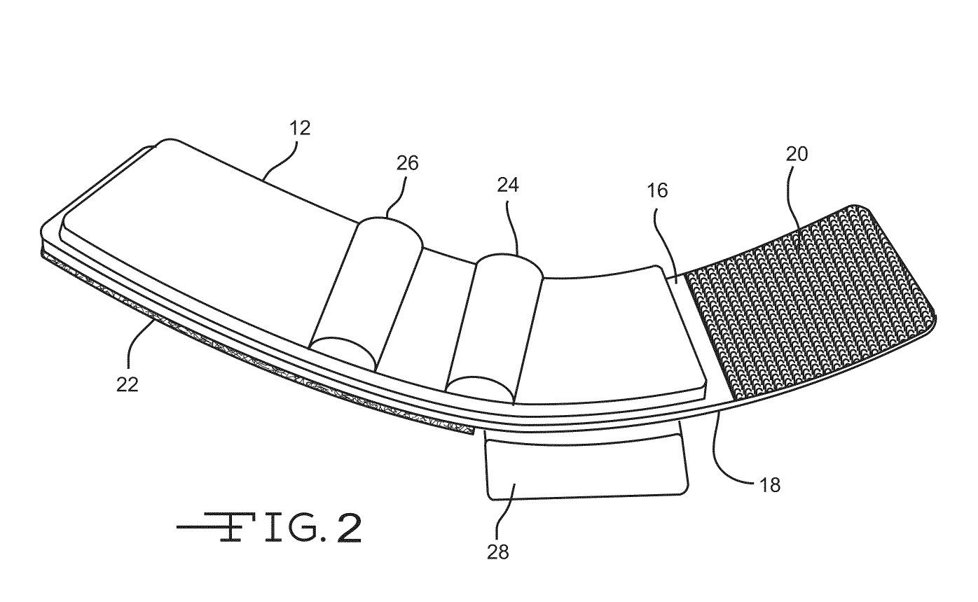

As shown in FIGS. 1 and 2, the occlusion cuff 12 has an interior surface 16 and an exterior surface 18. A first fastener 20 is positioned on the interior surface 16 and a second fastener 22 is positioned on the exterior surface 18. For example, the first and second fasteners 20 and 22 can be hook and loop-type fasteners that adhere to each other when pressed together. The engagement of the first and second fasteners 20 and 22 position the occlusion cuff 12 on a patient's arm.

Still referring to FIGS. 1 and 2, the interior surface 16 of the occlusion cuff 12 includes a first inflatable portion 24 and a second inflatable portion 26. The first and second inflatable portions 24 and 26 are configured to expand and contract in order to occlude or release one or both of the radial and ulnar arteries when the occlusion cuff 12 is positioned on a patient's arm as shown in FIG. 1.

As shown in FIG. 1, the occlusion cuff 12 has a control member 28 positioned on the exterior surface 18. In an embodiment, the control member 28 includes an energy source such as a battery to actuate the first and second inflatable portions 24 and 26, a cuff display screen 30 powered by the battery, and a cuff on/off button 32. The control member 28 includes hardware, software and/or firmware configured to control the operation of the radial check device 10.

Still referring to FIG. 1, the pulse oximeter 14 includes first and second finger members 34 and 36 that are connected by a hinge or other device to allow for clamping on a patient's finger. In an embodiment, the pulse oximeter 14 includes an energy source such as a battery, a pulse oximeter display screen 38 powered by the battery, and a pulse oximeter on/off button 40. The pulse oximeter 14 includes hardware, software and/or firmware configured to control the operation of the radial check device 10.

As shown in FIG. 1, the pulse oximeter 14 is operatively connected to the control member 28 as indicated by the line 42. For example, such connection can be wired or wireless. This allows for the transmission of signals between the pulse oximeter 14 and the control member 28.

Still referring to FIG. 1, the radial check device 10 includes a recording device 44 that is operatively connected to the control member 28 as indicated by the line 46. For example, such connection can be wired or wireless. This allows for the transmission of signals between the control member 28 and the recording device 44. The recording device 44 is used to produce a record of the data being generated by the radial check device 10. For example, the recording device 44 can be a printing device that produces a paper record of the data. In another example, the recording device 44 can be an electronic device such as a computer that produces an electronic record of the data. In another example, the recording device 44 is integral with the control member 28. The record can then be entered in a patient's paper and/or electronic chart to document a test.

In use, the radial check device 10 is positioned on a patient as shown in FIG. 1. In an embodiment, the first and second inflatable portions 24 and 26 are inflated to occlude the radial and ulnar arteries, respectively. The pulse oximeter 14 provides an automated oximetry tracing to the control member 28. In an embodiment, such tracing is shown on the cuff display screen 30. The second inflatable portion 26 is then released and the oximetry tracing is transmitted to the recording device 44. The record produced by the recording device 44 is then entered in the patient's chart. In an embodiment, the radial check device 10 can perform a reverse Barbeau test to check for radial flow for patients with repeat radial procedures.

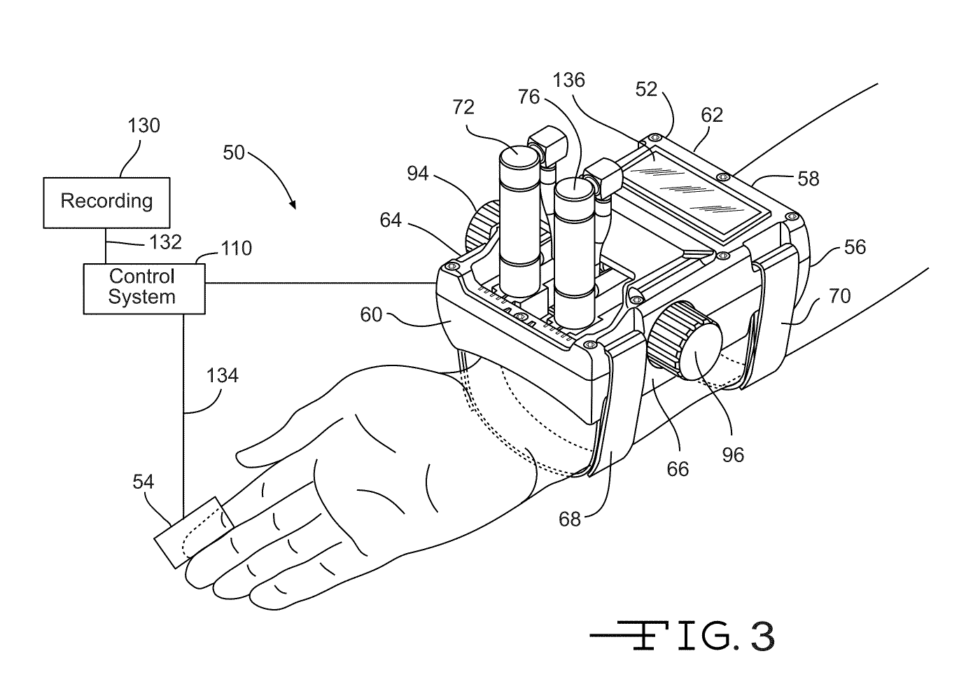

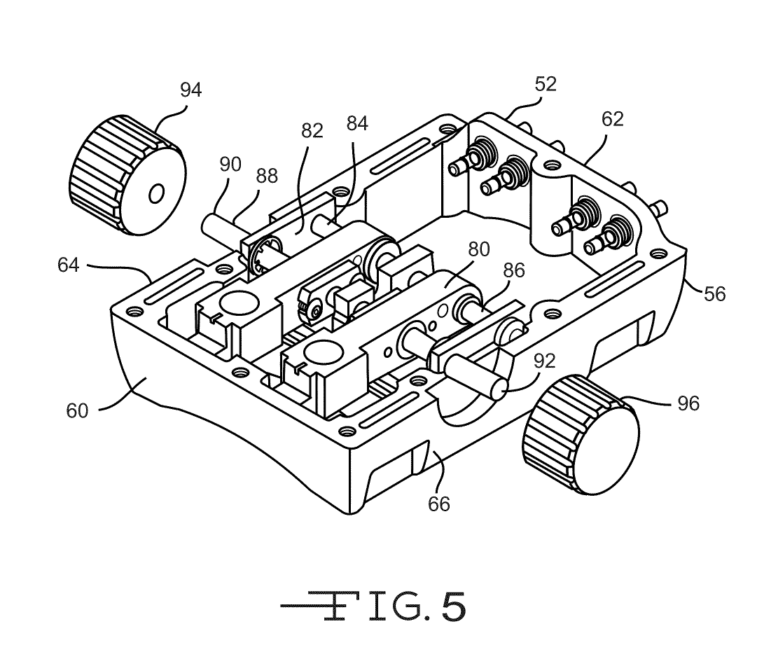

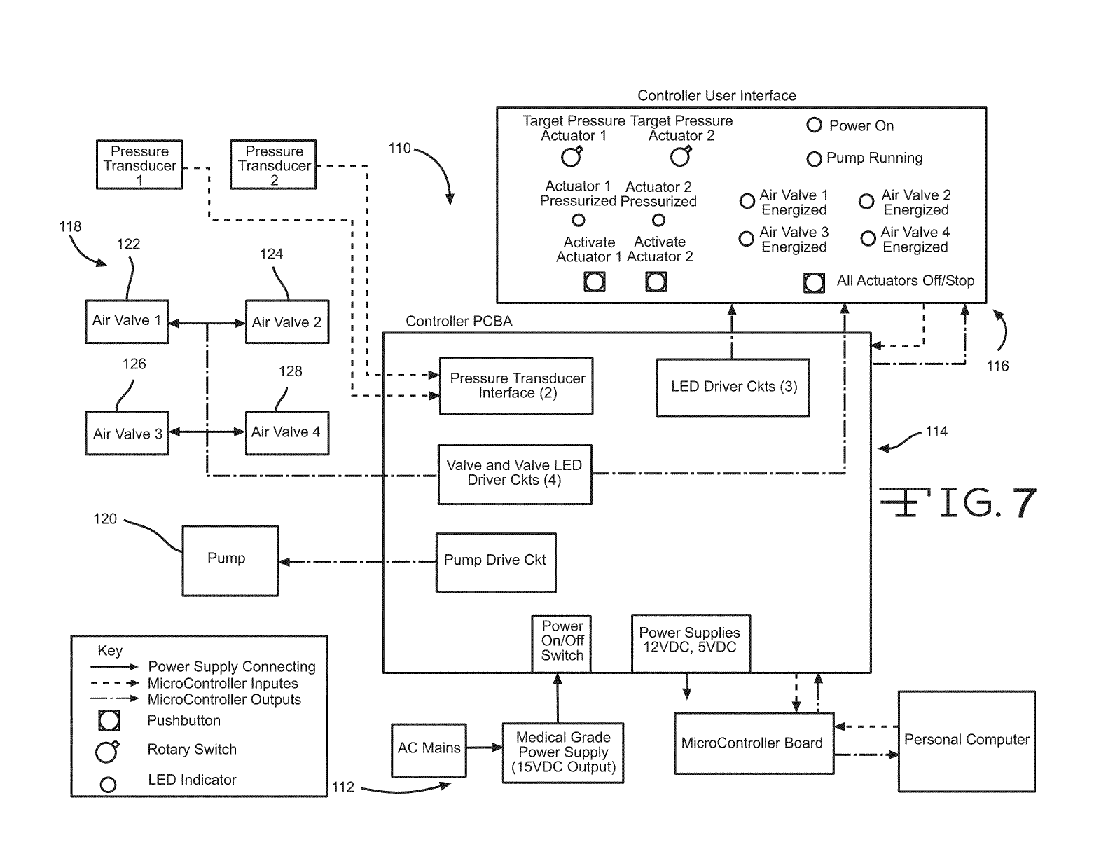

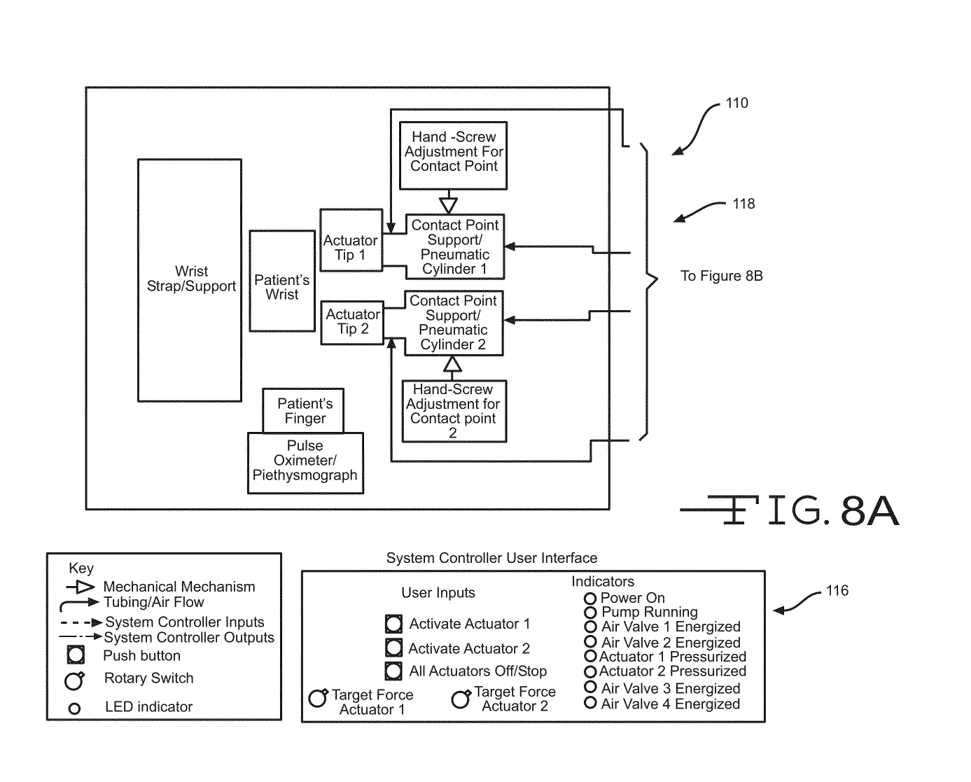

Referring to FIGS. 3-8B, an alternative embodiment radial check device 50 is shown and described. As shown in FIG. 3, the radial check device 50 has an occlusion cuff 52 configured for positioning on a patient's arm and a pulse oximeter 54 configured for positioning on a patient's finger, such as an index finger, or thumb. The occlusion cuff 52 has a bottom housing 56, a top housing 58, a first end 60, a second end 62, a first side 64 and a second side 66. A first strap 68 is positioned adjacent to the first end 60 and a second strap 70 is positioned adjacent to the second end 62. For example, the first and second straps 68 and 70 can include hook and loop-type fasteners that adhere to each other when pressed together. The first and second straps 68 and 70 position the occlusion cuff 52 on a patient's arm. The pulse oximeter 54 can be of the type described above with respect to pulse oximeter 14. In an embodiment, the pulse oximeter can be of a conventional type.

Referring to FIGS. 3-6, the occlusion cuff 52 includes a first actuator 72 having a first actuator tip 74 and a second actuator 76 having a second actuator tip 78. For example, as shown in this embodiment, the first and second actuators 72 and 76 can be pneumatic cylinder actuators. However, it should be understood that the first and second actuators 72 and 76 can be any suitable type of actuator that can move the first and second actuator tips 74 and 78.

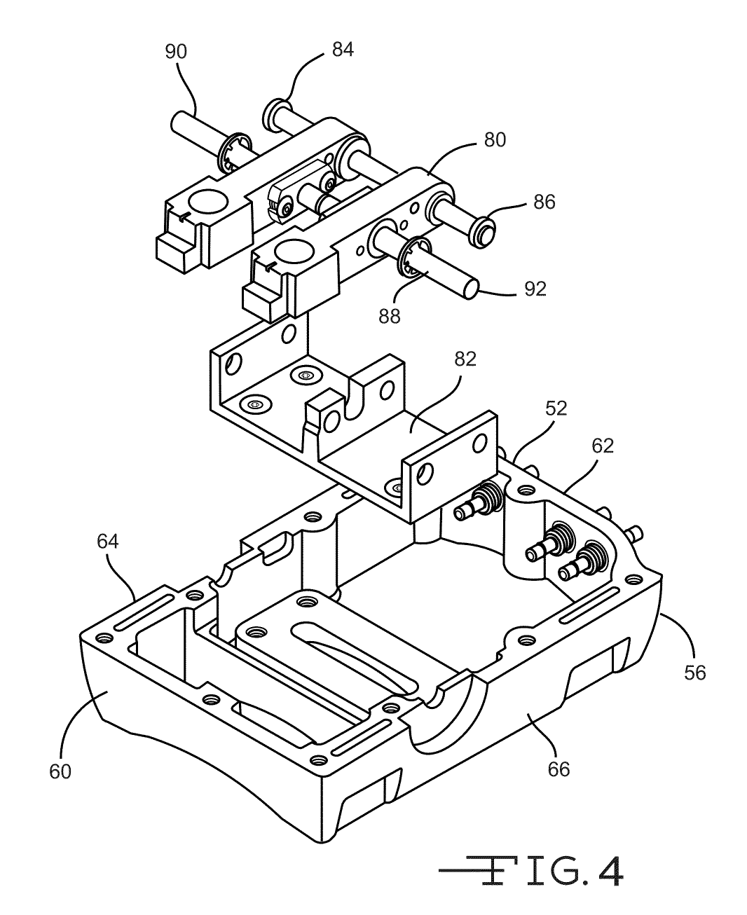

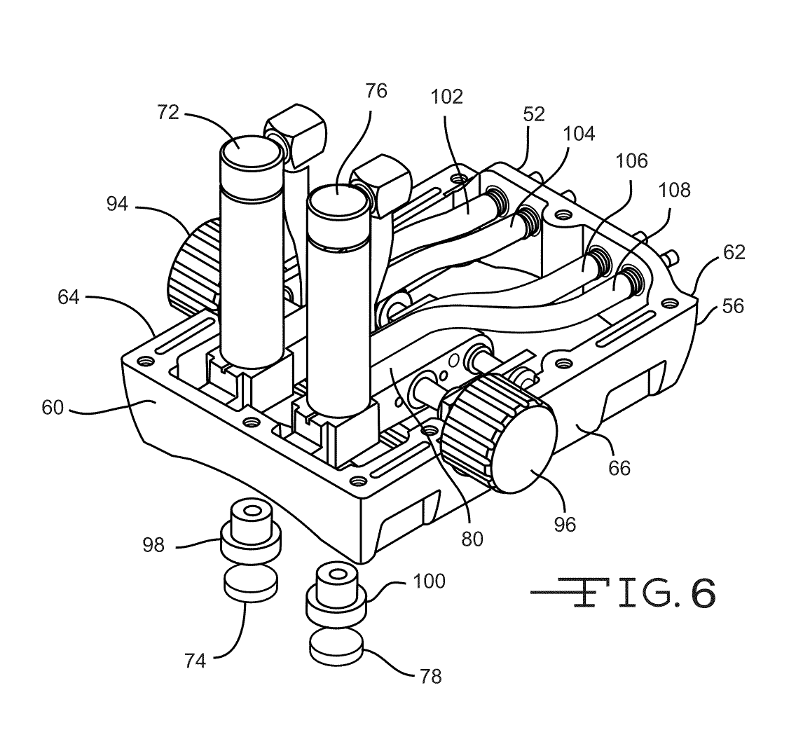

As shown in FIGS. 4-6, the first and second actuators 72 and 76 are movably mounted in the bottom housing 56 of the occlusion cuff 52. In this regard, the first and second actuators 72 and 76 are mounted on an actuator plate 80 that is pivotally positioned on a mounting frame 82 by first and second shafts 84 and 86. The actuator plate 80 includes a rod 88 that includes first and second rod ends 90 and 92 for mounting first and second knobs 94 and 96, which are positioned on the first and second sides 64 and 66, respectively, of the occlusion cuff 52. The turning of the first and second knobs 94 and 96 causes the first and second actuators 72 and 76 to be moved in order to adjust the first and second actuator tips 74 and 78 with respect to the patient's arm. As shown in FIG. 6, the first and second actuator tips 74 and 78 are positioned on first and second tip housings 98 and 100, respectively. As shown in FIG. 6, the first actuator 72 is in communication with first actuator tubes 102 and 104, and the second actuator 76 is in communication with second actuator tubes 106 and 108.

Referring to FIGS. 3, 7, 8A and 8B, the radial check device 50 has a control system 110. As shown and described in the drawings, the control system 110 generally includes a power supply 112, a controller 114, a controller interface 116, and an actuator control system 118. The control system 110 includes hardware, software and/or firmware configured to control the operation of the radial check device 50.

As shown in FIGS. 6, 7, 8A and 8B, the actuator control system 118 includes a pump 120 in communication with first, second, third and fourth valves 122, 124, 126 and 128 that are in communication with the first and second actuator tubes 102, 104, 106 and 108. The actuation of the pump 120 controls the actuation of the first and second actuators 72 and 76. It should be understood that any suitable control system 110 can be used to control the actuation of the first and second actuators 72 and 76.

Referring to FIG. 3, the radial check device 50 can include a recording device 130 that is operatively connected to the control system 110 as indicated by the line 132. For example, such connection can be wired or wireless. This allows for the transmission of signals between the control system 110 and the recording device 130. The recording device 130 is used to produce a record of the data being generated by the radial check device 50. For example, the recording device 130 can be a printing device that produces a paper record of the data. In another example, the recording device 130 can be an electronic device such as a computer that produces an electronic record of the data. In another example, the recording device 130 is integral with the control system 110. Further, the pulse oximeter 54 can be operatively connected to the control system 110, as indicated by the line 134, for recording data. The recorded data can then be entered in a patient's paper and/or electronic chart. As shown in FIG. 3, the occlusion cuff 52 can include a cuff display screen 136 to allow for the viewing of, for example, generated data and user interface symbols.

In use, the radial check device 50 is positioned on a patient as shown in FIG. 3. In this regard, the first and second actuators 72 and 76 are positioned over, for example, the radial and ulnar arteries of the patient. The positioning of the first and second actuators 72 and 76 can be adjusted by turning the first and second knobs 94 and 96. Once the radial check device 50 is in position, first and second actuators 72 and 76 are actuated or inflated by the control system 110 to cause the first and second actuator tips 74 and 78, respectively, to occlude the radial and ulnar arteries. The pulse oximeter 54 provides an oximetry tracing. The second actuator 76 is then released and the pulse oximeter 54 provides another oximetry tracing. The oximetry tracings are read by the healthcare professional performing the test. In an embodiment, the oximetry tracings are shown on the cuff display screen 136. In an embodiment, the oximetry tracings are transmitted to the recording device 130. In an embodiment, the record produced by the recording device 130 is entered in the patient's chart. In an embodiment, the radial check device 50 can perform a reverse Barbeau test to check for radial flow for patients with repeat radial procedures.

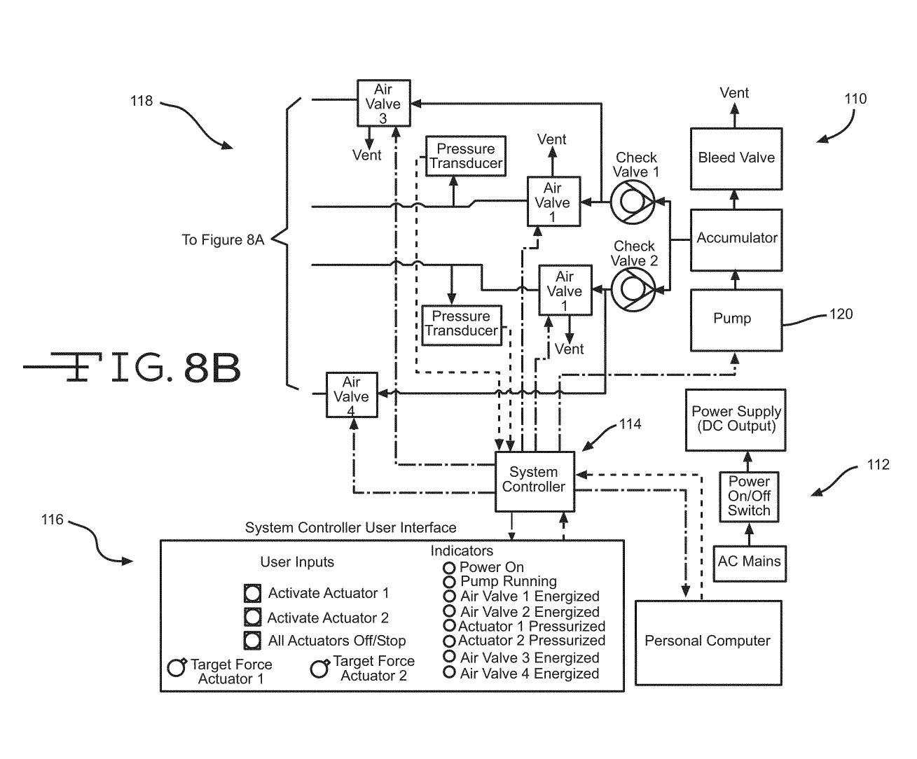

Referring to FIGS. 9-11, an alternative embodiment radial check device 150 is shown and described. As shown in FIG. 9, the radial check device 150 has an occlusion cuff 152 configured for positioning on a patient's arm and a pulse oximeter 154 configured for positioning on a patient's finger, such as an index finger, or thumb.

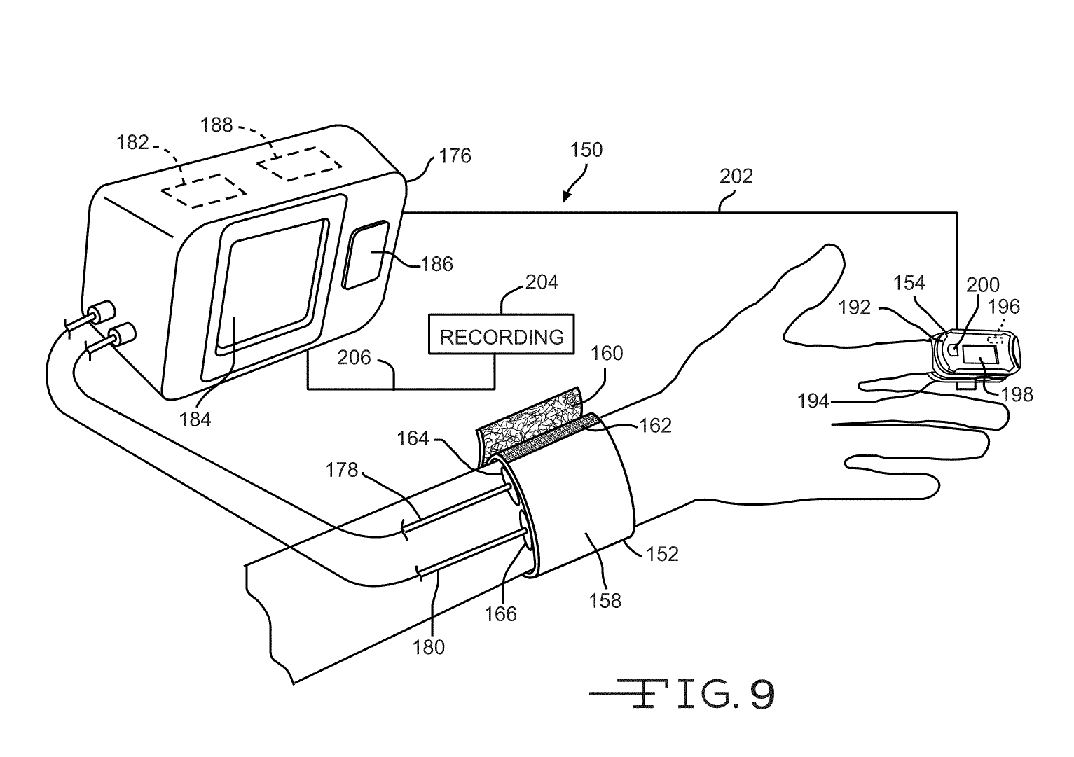

As shown in FIGS. 9 and 10, the occlusion cuff 152 has an interior surface 156 and an exterior surface 158. A first fastener 160 is positioned on the interior surface 156 and a second fastener 162 is positioned on the exterior surface 158. For example, the first and second fasteners 160 and 162 can be hook and loop-type fasteners that adhere to each other when pressed together. The engagement of the first and second fasteners 160 and 162 position the occlusion cuff 152 on a patient's arm.

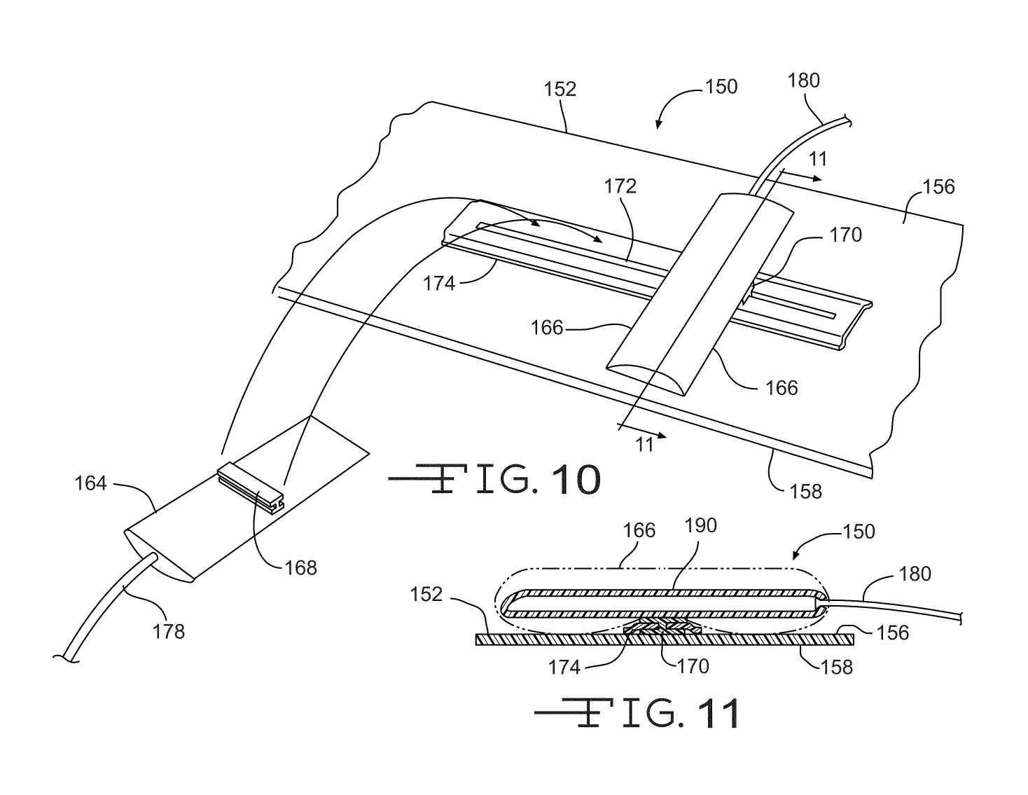

Referring to FIGS. 9-11, the interior surface 156 of the occlusion cuff 152 includes a first inflatable portion 164 and a second inflatable portion 166. The first and second inflatable portions 164 and 166 are configured to expand and contract in order to occlude or release one or both of the radial and ulnar arteries when the occlusion cuff 152 is positioned on a patient's arm as shown in FIG. 9. In an embodiment, the first and second inflatable portions 164 and 166 are adjustable with respect to the occlusion cuff 152 to allow such inflatable portions to be properly positioned with respect to the radial and ulnar arteries. For example, the first inflatable portion 164 can include a first portion mounting member 168 and the second inflatable portion 166 can include a second portion mounting member 170 in which such mounting members are sized and adapted for sliding engagement in a slot 172 of a mounting bracket 174 that is positioned on the interior surface 156 of the occlusion cuff 152 as shown in FIGS. 10 and 11. In addition to this example, it should be understood that the first and second inflatable portions 164 and 166 can be adjustably positioned with respect to the occlusion cuff 152 in a variety of ways. In an embodiment, the occlusion cuff 152 and the first and second inflatable portions 164 and 166 are constructed of plastic. In an embodiment, the occlusion cuff 152 and the first and second inflatable portions 164 and 166 are disposable after use.

As shown in FIG. 9, the radial check device 150 has a control member 176 that is separate from the occlusion cuff 152. In an embodiment, the control member 176 is operatively connected to the first and second inflatable portions 164 and 166 by first and second lines or tubes 178 and 180. In an embodiment, the control member 176 includes an energy source such as a battery 182 to actuate the first and second inflatable portions 164 and 166, a cuff display screen 184 powered by the battery 182, and a cuff on/off button 186. In an embodiment, the control member 176 includes an actuation device such as a pump system 188 to provide a fluid such as air through the first and second tubes 178 and 180 to inflate or deflate the first and second inflatable portions 164 and 166. In an embodiment, each of the first and second inflatable portions 164 and 166 includes an expandable and contractible bladder 190 that is in fluid communication with the first and second tubes 178 and 180 and thus the pump system 188 as shown in FIGS. 9 and 11. The control member 176 includes hardware, software and/or firmware configured to control the operation of the radial check device 150.

Referring to FIG. 9, the pulse oximeter 154 includes first and second finger members 192 and 194 that are connected by a hinge or other device to allow for clamping on a patient's finger. In an embodiment, the pulse oximeter 154 includes an energy source such as a battery 196, a pulse oximeter display screen 198 powered by the battery 196, and a pulse oximeter on/off button 200. The pulse oximeter 154 includes hardware, software and/or firmware configured to control the operation of the radial check device 150.

As shown in FIG. 9, the pulse oximeter 154 is operatively connected to the control member 176 as indicated by the line 202. For example, such connection can be wired or wireless. This allows for the transmission of signals between the pulse oximeter 154 and the control member 176.

Still referring to FIG. 9, the radial check device 150 includes a recording device 204 that is operatively connected to the control member 176 as indicated by the line 206. For example, such connection can be wired or wireless. This allows for the transmission of signals between the control member 176 and the recording device 204. The recording device 204 is used to produce a record of the data being generated by the radial check device 150. For example, the recording device 204 can be a printing device that produces a paper record of the data. In another embodiment, the recording device 204 can be an electronic device such as a computer that produces an electronic record of the data. In another example, the recording device 204 is integral with the control member 176. The record can then be entered in a patient's paper and/or electronic chart to document the test.

In use, the radial check device 150 is positioned on a patient as shown in FIG. 9. In an embodiment, the first and second inflatable portions 164 and 166 are inflated to occlude the radial and ulnar arteries, respectively. The pulse oximeter 154 provides an automated oximetry tracing to the control member 176. In an embodiment, such tracing is shown on the cuff display screen 184. The second inflatable portion 166 is then released and the oximetry tracing is transmitted to the recording device 204. The record produced by the recording device 204 is then entered in the patient's chart. In an embodiment, the radial check device 150 can perform a reverse Barbeau test to check for radial flow for patients with repeat radial procedures.

By way of non-limiting examples, the radial check device 10, 50, or 150 can be used in all places of a hospital or other healthcare facility (e.g., cardiac catheterization lab, vascular interventions lab, pediatric and/or adult intensive care unit, pre-operative anesthesia unit) in which the radial artery of a patient is used for cannulation. The invention provides a relatively simple medical device that a healthcare professional can utilize to access the ulnar and/or radial flow of a patient, and then place the documentation generated by the device in the paper and/or electronic chart of the patient. As it will be appreciated by those skilled in the art, the invention establishes a new standard of pre-procedural care for patients undergoing any type of radial artery canalization.

Certain embodiments of the apparatus disclosed herein are defined in various examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A radial check device comprising:
an occlusion cuff having first and second portions adapted to occlude and release the radial and ulnar arteries of a patient;
a pulse oximeter adapted to generate an oximetry tracing of the patient upon release of one of the arteries; and
a recording device operatively connected to the pulse oximeter to generate a record of the oximetry tracing.

2. The radial check device of claim 1, wherein the occlusion cuff has releasable fasteners.

3. The radial check device of claim 1, wherein the first and second portions are positioned on an interior surface of the occlusion cuff.

4. The radial check device of claim 1, wherein the first and second portions are adapted to expand and contract to occlude or release one or both of the radial and ulnar arteries.

5. The radial check device of claim 1, wherein the first and second portions include first and second actuators having first and second actuator tips, respectively, adapted to occlude or release one or both of the radial and ulnar arteries.

6. The radial check device of claim 1, wherein the first and second portions are adapted to be adjustable.

7. The radial check device of claim 1, wherein at least one of the first and second portions is adapted to be adjustable.

8. The radial check device of claim 1, wherein the radial check device has a control system adapted to actuate the first and second portions.

9. The radial check device of claim 1, wherein the occlusion cuff has a display screen.

10. The radial check device of claim 1, wherein the pulse oximeter has a display screen.

11. The radial check device of claim 1, wherein the pulse oximeter is operatively connected to the occlusion cuff.

12. The radial check device of claim 1, wherein the pulse oximeter is operatively connected to a control system, the control system being operatively connected to the recording device.

13. The radial check device of claim 1, wherein the recording device produces a paper record.

14. The radial check device of claim 1, wherein the recording device produces an electronic record.

15. A method of measuring ulnar flow comprising the steps of:
occluding the radial and ulnar arteries;
releasing the ulnar artery;
generating an oximetry tracing; and
producing a record of the oximetry tracing.

16. A method of measuring radial flow comprising the steps of:
occluding the radial and ulnar arteries;
releasing the radial artery;
generating an oximetry tracing; and
producing a record of the oximetry tracing.
